(19) United States
(12) Patent Application Publication
(10) Pub. No.: US 2006/0149266 A1
(43) Pub. Date: Jul. 6, 2006

(54) Anchor for screw fixation of soft tissue to bone

(75) Inventor: Frank A. Cordasco, New York, NY (US)

Correspondence Address: DARBY & DARBY P.C., P. O. BOX 5257, NEW YORK, NY, 10150-5257, US

(73) Assignee: New York Society for the Ruptured and Crippled Maintaining The Hospital for Special Surgery, New York, NY (US)

(21) Appl. No.: 11/298,239

(22) Filed: Dec. 7, 2005

Related U.S. Application Data
(60) Provisional application No. 60/635,303, filed on Dec. 10, 2004.

(51) Int. Cl. A61B 17/58

(52) U.S. Cl. 606/76

(57) ABSTRACT

The present invention relates to the field of orthopedics in addressing soft tissue to bone attachment. The embodiments of the present invention provide for a bioabsorbable inlay, a bioabsorbable inlay assembly, and a method to implant the bioabsorbable inlay between soft tissue and bone. Also provided is a means for facilitating soft tissue to bone attachment through the use of a bioabsorbable inlay, bounded with osteoconductive or osteoinductive materials, affixed at the interface of soft tissue to bone attachment sites. In an embodiment, the bioabsorbable inlay is formed into circular discs with holes for sutures to pass through and bounded with hydroxyapatite. The circular discs are then placed in communication between the soft tissue and bone at the appropriate site of attachment through the use of anchors and/or sutures. The bioabsorbable circular disc inlays are either overlaid or recessed into the bone.

### CROSS-REFERENCE TO RELATED APPLICATIONS

The present application claims priority to Provisional Application No. 60/635,303 filed Dec. 10, 2004, the disclosure of which is incorporated by reference.

### BACKGROUND OF THE INVENTION

1. Field of the Invention

The present invention relates to a device and method for treating repairs of soft tissue to bone. More specifically, the present invention relates to facilitating the healing process of soft tissue, e.g., tendons or ligaments, to bone.

2. Background

Complete healing of tendons or ligaments to bone may take up to about six months after a soft tissue to bone repair. As such a patient, who has undergone a soft tissue to bone repair, may not resume full function and/or range of motion for up to about six months.

An example of soft tissue to bone repair is rotator cuff repairs of the shoulder. The rotator cuff may need to be surgically repaired if the tendon is torn. Typical procedures to repair rotator cuff tears are done with metal or bioabsorbable anchors and sutures, where the suture is used to stitch and secure the tendon to the bone. However, studies have shown that approximately 30 to 90% of patients who have undergone traditional rotator cuff repairs, i.e., suture and anchor procedures, have persistent defects or tears of the rotator cuff.

Additional devices and techniques have been developed that allow for such repairs to be conducted without the use of sutures. These devices typically employ the use of bioabsorbable anchors with a capped head to secure the soft tissue to bone with direct compression applied through the capped head feature. The problem with these anchors is that the capped heads tend to interfere with the normal biomechanical motion of the shoulder. This interference may cause the acromion to impinge upon the capped head and lead to failure or fracture of the cap on the anchors.

Osteoconductive materials are bioactive agents that have been used within the orthopedic and dental fields to promote bone growth. These materials, such as hydroxyapatite or tricalcium phosphate, have been applied to orthopedic and dental implants to promote bone growth onto or into the implants. However, these osteoconductive materials have not yet been successfully used to promote tendon to bone formation or ligament to bone formation.

Osteoinductive materials are bioactive agents that have recently become of great interest in the orthopedic field as a method of inducing bone formation. Osteoinductive materials differ from osteoconductive materials in that an osteoconductive material promotes bone growth due to its proximity to bone. In contrast, an osteoinductive material will induce bone formation in the absence of any surrounding bone source. However, difficulties arise in applying osteoinductive materials, such as bone morphogenic proteins or osteogenic proteins, for soft tissue repairs. Such difficulties include the lack of control of the osteoinductive properties of these compounds, which properties can result in excessive bone formation at the site of implantation.

Accordingly, a need exists for a bioabsorbable device that would promote the healing of tendon to bone or ligament to bone.

### BRIEF SUMMARY OF THE INVENTION

The present invention provides for a bioabsorbable inlay, a bioabsorbable inlay assembly, and a method to implant the bioabsorbable inlay between soft tissue and bone. The present invention also provides a means for facilitating soft tissue to bone healing in shorter time than with typical suture and anchoring techniques through the use of an implantable bioabsorbable bioactive inlay. The bioabsorbable inlay can be formed into circular discs, bounded with a bioactive agent, such as an osteoconductive or osteoinductive material, and contain holes for sutures to pass through. The circular discs are then affixed through the use of anchors and/or sutures between the soft tissue and bone at the appropriate site of attachment. The circular discs can be overlaid onto or recessed into the bone.

### BRIEF DESCRIPTION OF THE SEVERAL VIEWS OF THE DRAWING(S)

Further aspects and advantages of the invention will become apparent from the following description and claims, and from the accompanying drawings, wherein:

FIG. 1 is a perspective view of a circular bioabsorbable inlay disc connected to a suture anchor with sutures;

FIG. 2 is an anterior cross sectional view of a bioabsorbable inlay disc as applied to a rotator cuff of a right shoulder;

FIG. 3 is a superior cross sectional view of a plurality of bioabsorbable inlay discs secured between soft tissue and bone of the rotator cuff;

FIG. 4 is a top plan view of an annulus bioabsorbable inlay disc;

FIG. 5 is a cross sectional view of a suture anchor inserted into the bone flush with the bone surface;

FIG. 6 is a cross sectional view of a suture anchor inserted into the bone and sitting proud of the bone surface;

FIG. 7 is a perspective view of a headed suture anchor and annulus inlay disc assembly;

FIG. 8 is a flowchart of a method of the present invention; and

FIG. 9 is a flowchart of another method embodiment of the present invention.

### DETAILED DESCRIPTION OF THE INVENTION

The present invention provides for a bioabsorbable inlay, a bioabsorbable inlay assembly, and method to implant the bioabsorbable inlay between soft tissue and bone. The present invention also provides a method for facilitating soft tissue to bone repairs through the use of a bioabsorbable bioactive inlay implanted between soft tissue and bone at the site of attachment.

Applications of the present invention for treating soft tissue to bone repairs can include the following soft tissues: biceps femoris tendons, biceps tenodesis, biceps tendon reattachment, capsular shift or capsulolabral reconstruction in the shoulder, deltoid repair, iliotibial band tendons, iliotibial band tendodesis, lateral collateral ligaments, lateral ankle stabilization, medial collateral ligaments, patellar tendons, pectoralis major tendons, posterior cruciate ligaments (at the tibial side), rotator cuff tendons, rotator cuff repair, tennis elbow repair, and triceps tendons. Detailed descriptions of these soft tissues are further described in H. Gray et al., Gray's Anatomy (38th ed. 1995).

All soft tissues applicable to the present invention can be classified as either tendons or ligaments. Tendons are a band of tough, inelastic fibrous tissue that connects a muscle with its bony attachment. Ligaments are a sheet or band of tough, fibrous tissue supporting an organ or connecting bones or cartilages at a joint.

A bioabsorbable polymeric matrix of the present invention can be selected from a variety of bioabsorbable polymers. Such bioabsorbable polymers may include Poly (L-lactic acid) (PLLA), Poly (D, L-lactide) (PDLLA), Polylatic acid copolymers, Poly (Glycolic acid) (PGA), elastomeric hydrogels, collagen or any combination thereof. Other suitable bioabsorbable polymers are mentioned in U.S. Pat. No. 4,968,317 and J. Middleton et al., Med. Plastics Biomater., March 1998. Bioabsorbable materials are advantageous because they would allow the soft tissue to attach completely with the bone without an intervening layer of material at the site of attachment. These polymers may be absorbed by the body over several weeks or months and potentially replaced by natural bone. Bioabsorbable polymers with a slow degradation rate are preferred for soft tissue to bone repairs due to the slow healing time associated with such orthopedic procedures. Soft tissue to bone repairs may take up to about six months or more to completely heal.

The bioabsorbable polymeric matrix is preferably formed from PLLA or PDLLA due to the materials slow degradation rate in vivo.

The bioabsorbable polymeric matrix can be formed into any shape such as a circular disc, hemi-circular disc, square disc, rectangular disc, ellipsoid disc, cylinder, annulus, cube, etc. In a preferred embodiment, the bioabsorbable polymeric matrix is formed into a circular disc 10 as shown in FIG. 1, because this shape can be easily inserted between the soft tissue 20 and bone and maximizes the surface area for bone, tendon, or ligament contact per disc. A circular disc shape can also be easily recessed into the bone without the need to properly orient the disc, as compared with a square disc configuration. Furthermore, the bone preparation for a recessed circular disc is simpler then with any other shape, such as a square. A single step reaming of the bone with a corresponding sized circular reamer, to the appropriate depth, can adequately complete the bone preparation for a recessing circular inlay. Any other shape with corners, e.g., a square, may require several reaming steps or bone cut steps to complete the bone preparation for a recessed inlay device. The circular discs can range in size and thickness depending upon the nature of the procedure. In a preferred embodiment, the circular disc shaped inlay is about six to ten mm in diameter and from about three to five mm thick.

The bioabsorbable inlay can be formed with one or more holes 12 as a receptor for receiving an affixing member, such as sutures (for example #2 or #3 multifilament sutures), staples, or bone anchors, to affix the inlay between soft tissue and bone. Preferably, no more then about four holes should be incorporated into the inlay due to the additional mechanical stresses added to the inlay as a result of the holes. The position of the holes for sutures can vary anywhere along the surface 14 of the inlay. The positioning of the holes can be symmetric, asymmetric, anatomically positioned or made intraoperatively by the user. The size of the hole need only be sufficient to allow sutures 16 to pass.

Bioactive osteoconductive materials that can be used in accordance with the invention may include hydroxyapatite (HA), tricalcium phosphate (TCP), or a combination thereof. The osteoconductive material can be bound to the bioabsorbable polymeric matrix by either impregnating the polymeric matrix and/or coating the polymeric matrix. Various methods of producing these impregnated polymeric matrix compositions can include hot pressing, molding, or through a biomimetic approach (see e.g., R. Zhang et al., Macromol. Biosci., 4(2), February 2000, p. 100-111; S. M. Rea et al., J. Mater. Sci. Mater. Med., 15(9), September 2004, p. 997-1005; N. Ignjatovic et al., J. Biomed. Mater. Res. Nov. 15, 2004 71B(2), p. 284-294; A. Matsuda et al., J. Mater. Sci. Mater. Med., 14(11), November 2003, p. 973-8; M. Wang et al., J. Mater. Sci. Mater. Med., 12(9), September 2001, p. 821-826). Whereas, coating methods for these bioabsorbable polymeric matrixes can include such methods as precipitation deposition, biomimetic processes electrolytic deposition, or electrophoretic processes. Impregnating the polymeric matrix with an osteoconductive material is preferable to coatings as this would allow for constant exposure of the osteoconductive material throughout the degradation of the inlay in vivo. A bioabsorbable polymeric matrix is required in contrast to a pure HA or TCP inlay, as a pure HA or TCP inlay would be absorbed by the body too quickly to promote effective soft tissue to bone formation.

Bioactive osteoinductive materials that can be used in accordance with the present invention can include bone morphogenic proteins (BMP), osteogenic proteins (OP), or combinations thereof. Osteoinductive materials may be applied manually to the bioabsorbable polymeric matrix at the time of surgery by the end user. A bioabsorbable polymeric matrix is required as BMPs or OPs require a substrate as a carrier to effectively initiate osteoinduction.

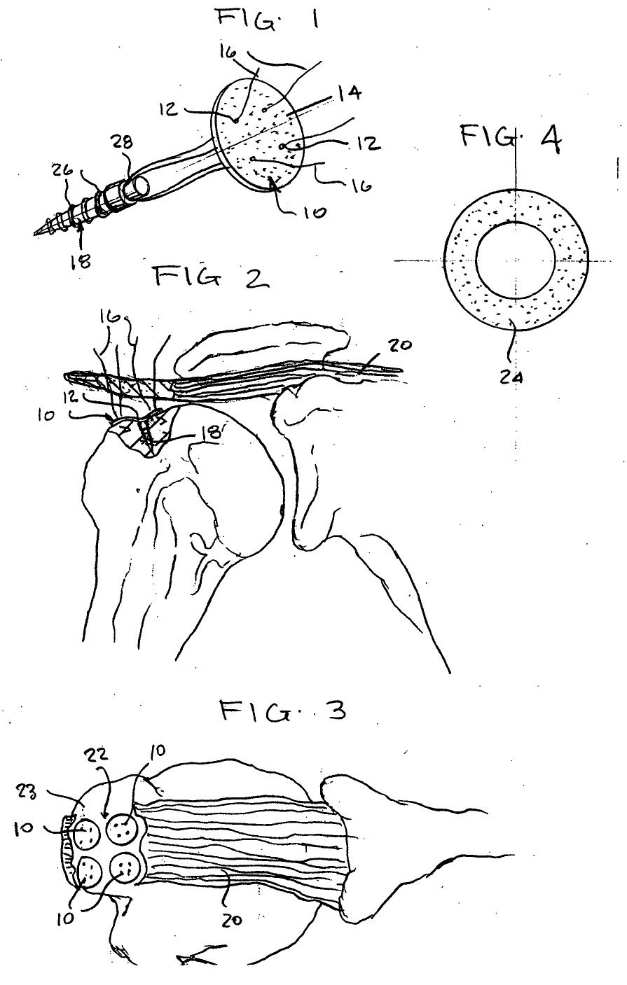

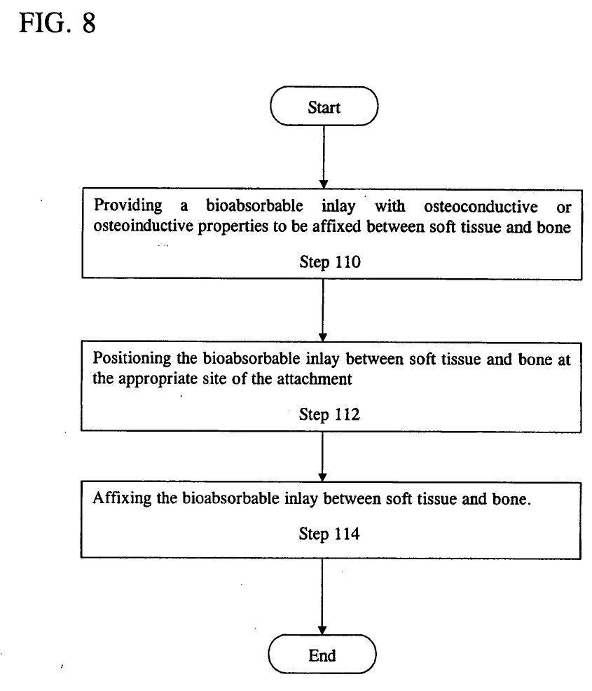

The present invention also provides a method for promoting soft tissue to bone attachment, see FIGS. 2 and 8. This method calls for providing a bioabsorbable inlay 10 with osteoconductive or osteoinductive properties to be affixed between soft tissue 20 and bone 32, Step 110. Another step calls for positioning the bioabsorbable inlay 10 between soft tissue 20 and bone 32 at the appropriate site of soft tissue to bone attachment, Step 112. Yet another step calls for affixing the bioabsorbable inlay 10 between soft tissue 20 and bone 32, Step 114.

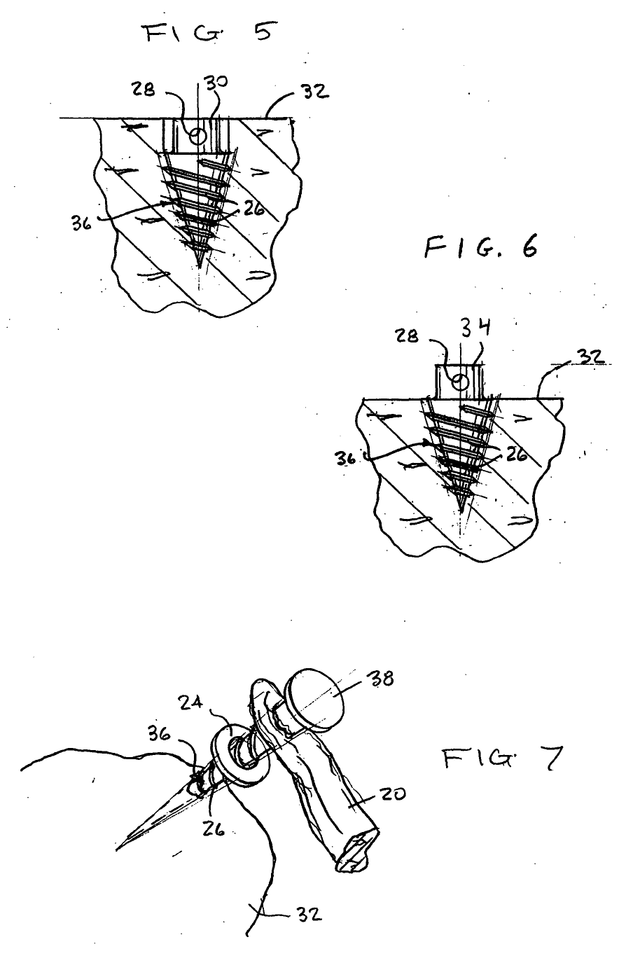

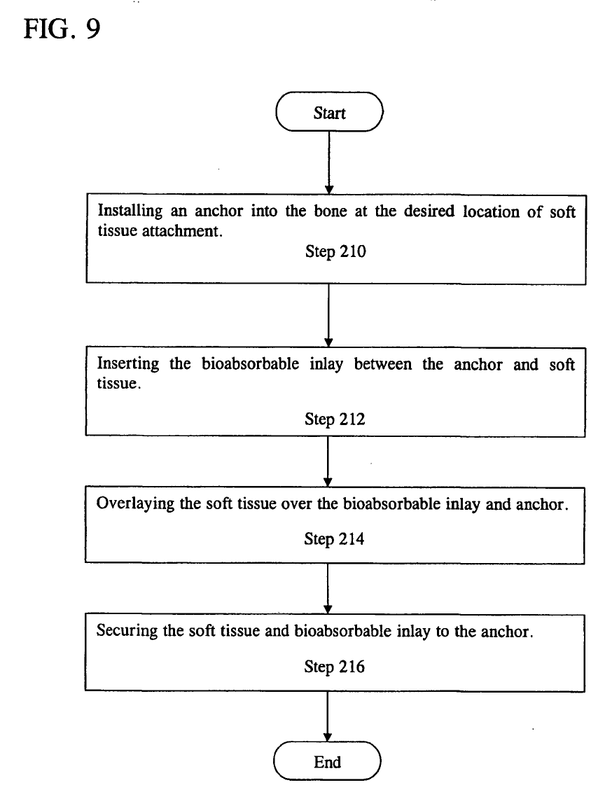

The present invention further provides a method for affixing the bioabsorbable inlay in between soft tissue and bone as illustrated in FIG. 2 and FIG. 9. This method calls for installing a suture anchor 18 into the bone at the desired location of soft tissue attachment, Step 210. A suture anchor 18 is a commercially available device that is capable of being threaded or impacted into the bone to provide a secure attachment site for sutures. Suture anchors typically consist of threads 26 and an engagement site 28 for sutures to attach. FIG. 2 illustrates a suture anchor screw 18 inserted into the superior aspect of the right humerus. Suture anchors can be inserted into the bone by pre-drilling the site or by using a self-tapping device. However, alternate suture anchor designs can be inserted without pre-drilling but requiring only direct impaction. The depth in which suture anchors are inserted depends upon the end user preferences. However, suture anchors can be inserted to sit flush 30 with the bone surface 32 or to sit proud 34 of the bone surface 32, see FIGS. 5 and 6. Typical commercially available suture anchors are provided with sutures pre-assembled.

Another step calls for inserting the bioabsorbable inlay 10 between the anchor and soft tissue 20, Step 212. Additionally, the bone at the desired location of soft tissue attachment may be reamed or resurfaced to allow the inlay to be recessed 30 and sit flush with the bone surface 32.

Another step calls for overlaying the soft tissue 20 over the bioabsorbable inlay 10 and suture anchor 18, Step 214.

Yet another step calls for securing the soft tissue 20 and bioabsorbable inlay 10 to the suture anchor 18, Step 216. This can be accomplished with one or more sutures 16. Suturing encompasses securing the soft tissue 20 and bioabsorbable inlay 10 to a suture anchor 18 by stitching the entire assembly together. The suturing of the soft tissue 20 and bioabsorbable inlay 10 to the suture anchor 18 may be completed with a simple mattress or modified Mason-Allen stitch technique.

In a preferred embodiment, the bioabsorbable inlay 10 is positioned to cover as much of the footprint 22 of the soft tissue to bone interface as possible, see FIG. 3. This can be accomplished with one or more inlays depending on the size of the soft tissue point of attachment.

The methods of the present invention can be accomplished arthroscopically or with an open procedure.

The bioabsorbable inlay can be used in combination with any number of commercially available suture anchors, for example Stryker®'s Xcel suture anchor, Mainstay® suture anchor, Arthrotek®'s Collared Harpoon® Anchor or Corkscrew anchor, or Smith and Nephew's TwinFix Absorbable suture anchor. Such suture anchors are made of metal or bioabsorbable materials.

In an alternative embodiment, the bioabsorbable inlay, bounded with an osteoconductive material, can by formed into an annulus disc 24, as shown in FIG. 4, and used in combination with headed tissue anchors 36 or interference screws, see FIG. 7. As shown in FIG. 7, a headed tissue anchor 36 does not use sutures to secure the soft tissue to bone, but applies compression of the soft tissue to bone through its headed cap configuration 38. As such, an annulus shaped disc inlay 24 can be applied to a headed tissue anchor 36 with the inlay positioned between the soft tissue 20 and bone surface 32 and with the threads of the headed tissue anchor passing through the center of the inlay 24; much like a washer is used in a nut and bolt configuration. Similarly, the annulus disc formation may also be used with interference screws in a like manner.

The embodiments of the present invention are shown and described for purposes of illustration only and not for purposes of limitation. Many variations of the present invention will suggest themselves to those skilled in the art in light of the above detailed description. All such obvious variations are within the fully intended scope of the application.

The above mentioned patents, applications, test methods, and publications are hereby incorporated by reference in their entirety.

What is claimed is:

1. A bioabsorbable inlay for promoting soft tissue to bone attachment comprising:
a bioabsorbable polymeric matrix;
a bioactive agent bound to the bioabsorbable polymeric matrix; and
a receptor for receiving an affixing member for affixing the bioabsorbable inlay between soft tissue and bone.

2. The bioabsorbable inlay of claim 1, wherein the bioabsorbable polymeric matrix is Poly (L-lactic acid) (PLLA), Poly (D,L-lactide) (PDLLA), Polylatic acid copolymer, Poly (Glycolic acid) (PGA), elastomeric hydrogel, collagen or a combination thereof.

3. The bioabsorbable inlay of claim 1, wherein the bioactive agent is hydroxyapatite (HA), tricalcium phosphate (TCP), bone morphogenic protein (BMP), osteogenic protein (OP), or a combination thereof.

4. The bioabsorbable inlay of claim 1 wherein the receptor is a hole.

5. The bioabsorbable inlay of claim 1, wherein the inlay is from about 3 to 5 mm in thickness.

6. The bioabsorbable inlay of claim 1, wherein the inlay is shaped as a circular disc or annulus.

7. The bioabsorbable inlay of claim 6, wherein the inlay is from about 6 mm to 10 mm in diameter.

8. The bioabsorbable inlay of claim 1, wherein the inlay contains a hole for sutures to pass through.

9. The bioabsorbable inlay of claim 8, wherein the number of holes is from about 2 to 4 holes.

10. The bioabsorbable inlay of claim 1, wherein the inlay bioabsorbes after about 6 weeks.

11. The bioabsorbable inlay of claim 1, wherein the soft tissue is, or associated with, biceps femoris tendons, biceps tenodesis, biceps tendon reattachment, capsular shift or capsulolabral reconstruction in the shoulder, deltoid repair, iliotibial band tendons, iliotibial band tendodesis, lateral collateral ligaments, lateral ankle stabilization, medial collateral ligaments, patellar tendons, pectoralis major tendons, posterior cruciate ligaments (at the tibial side), rotator cuff tendons, rotator cuff repair, tennis elbow repair, triceps tendons, or a combination thereof.

12. A bioabsorbable inlay configured to be affixed between soft tissue and bone to promote attachment of the soft tissue to the bone.

13. The bioabsorbable inlay of claim 12 wherein the configuration is of a circular disc having a hole.

14. The bioabsorbable inlay of claim 12 wherein the configuration is of an annulus.

15. A method of promoting soft tissue to bone attachment comprising the steps of:
providing a bioabsorbable inlay comprising, a bioabsorbable polymeric matrix, a bioactive agent bound to the bioabsorbable polymeric matrix, and a receptor for receiving an affixing member to the bioabsorbable inlay between soft tissue and bone;
positioning the bioabsorbable inlay between soft tissue and bone at a site of attachment; and
affixing the bioabsorbable inlay between soft tissue and bone.

16. The method of claim 15, wherein the step of affixing the bioabsorbable inlay between soft tissue and bone, comprises the steps of:
installing an anchor into the bone at the desired location of soft tissue attachment;
inserting the bioabsorbable inlay between the anchor and soft tissue;
overlaying the soft tissue over the bioabsorbable inlay and anchor; and
securing the soft tissue and bioabsorbable inlay to the anchor.

17. The method of claim 16, wherein the bone at the desired location of soft tissue attachment is rasped or reamed to allow the bioabsorbable inlay to lay recessed and sit flush with the bone surface.

18. The method of claim 16, wherein the method is conducted arthroscopically.

19. The method of claim 16, wherein the method is conducted with an open technique.

20. The method of claim 16, wherein a plurality of bioabsorbable inlays is used to replicate the footprint of the soft tissue to bone interface.

21. The method of claim 16, wherein the securing step is selected from a group consisting of suturing, stitching, screwing, compressing, stapling, or gluing.

22. A bioabsorbable inlay assembly for promoting soft tissue to bone attachment, the assembly comprising:
a bioabsorbable inlay comprising a bioabsorbable polymeric matrix, a bioactive agent bound to the bioabsorbable polymeric matrix, and a receptor for receiving an affixing member for affixing the bioabsorbable inlay between soft tissue and bone; and
an anchor for inserting into the bone at the location of soft tissue attachment, the anchor in communication with the bioabsorbable inlay; and
a suture for securing the soft tissue, bioabsorbable inlay, and anchor together.

23. The bioabsorbable inlay assembly of claim 22, wherein the bioabsorbable polymeric matrix is Poly (L-lactic acid) (PLLA), Poly (D,L-lactide) (PDLLA), Polylatic acid copolymer, Poly (Glycolic acid) (PGA), elastomeric hydrogel, collagen, or combination thereof.

24. The bioabsorbable inlay assembly of claim 22, wherein the bioactive agent is hydroxyapatite (HA), tricalcium phosphate (TCP). bone morphogenic protein (BMP), an osteogenic protein (OP), or a combination thereof.

25. The bioabsorbable inlay assembly of claim 22, wherein the receptor is a hole.

26. The bioabsorbable inlay assembly of claim 22, wherein the inlay is from about 3 mm to about 5 mm in thickness.

27. The bioabsorbable inlay assembly of claim 22, wherein the inlay is circular in shape.

28. The bioabsorbable inlay assembly of claim 26, wherein the inlay is from about 6 mm to 10 mm in diameter.

29. The bioabsorbable inlay assembly of claim 22, wherein the inlay contains a hole for sutures to pass through.

30. The bioabsorbable inlay assembly of claim 29, wherein the number of holes is from about 2 to about 4 holes.

31. The bioabsorbable inlay assembly of claim 22, wherein the inlay bioabsorbes after about 6 weeks.

32. The bioabsorbable inlay assembly of claim 22, wherein the soft tissue is, or associated with, biceps femoris tendons, biceps tenodesis, biceps tendon reattachment, capsular shift or capsulolabral reconstruction in the shoulder, deltoid repair, iliotibial band tendons, iliotibial band tendodesis, lateral collateral ligaments, lateral ankle stabilization, medial collateral ligaments, patellar tendons, pectoralis major tendons, posterior cruciate ligaments (at the tibial side), rotator cuff tendons, rotator cuff repair, tennis elbow repair, triceps tendons, or combinations thereof.
